(19) United States
(12) Patent Application Publication
(10) Pub. No.: US 2002/0150601 A1
(43) Pub. Date: Oct. 17, 2002

(54) Use of folic acid and/or derivatives thereof for the preparation of cosmetic or dermatological preparations for the prophylaxis of damage to DNA intrinsic to the skin and/or for the repair of existing damage to DNA intrinsic to the skin

(75) Inventors: Heiner Max, Hamburg (DE); Katriu Will, Hamburg (DE); Ralph Schimpf, Bonningstedt (DE); Thomas Raschke, Hamburg (DE); Birgit Hargens, Hamburg (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(73) Assignee: Beiersdorf Aktiengesellschaft

(21) Appl. No.: 10/021,627

(22) Filed: Dec. 12, 2001

(30) Foreign Application Priority Data
Dec. 14, 2000 (DE) 100 62 401.4

(51) Int. Cl. A61K 31/525; A61K 7/00

(52) U.S. Cl. 424/401; 514/251

(57) ABSTRACT

Use of folic acid and/or derivatives thereof for the preparation of cosmetic or dermatological preparations for the prophylaxis of damage to DNA intrinsic to the skin and/or for the repair of existing damage to DNa intrinsic to the skin.

The present invention relates to the use of folic acid and/or derivatives thereof for the preparation of cosmetic or dermatological preparations for the prophylaxis of damage to DNA intrinsic to the skin and/or for the repair of existing damage to DNA intrinsic to the skin.

The skin is exposed to a large number of environmental influences which can lead to damage. As well as lipids and proteins, the DNA in particular can be affected.

The harmful effect of the ultraviolet part of solar radiation on the skin is generally known. Whereas rays with a wavelength below 290 nm (the UVC region) are absorbed by the ozone layer in the earth's atmosphere, rays in the range between 290 nm and 320 nm, the UVB region, cause erythema, simple sunburn or even burns of greater or lesser severity.

A maximum erythema activity of sunlight is given as the relatively narrow region around 308 nm.

Numerous compounds are known for protecting against UVB radiation; these are derivatives of 3-benzylidenecamphor, of 4-aminobenzoic acid, of cinnamic acid, of salicylic acid, of benzophenone and also of 2-phenylbenzimidazoles.

It is also important to have available filter substances for the range between about 320 nm and about 400 nm, the UVA region, since the rays in this range can cause reactions in cases of photosensitive skin. It has been found that UVA radiation leads to damage of the elastic and collagenous fibers of connective tissue, which leads to premature aging of the skin and is to be regarded as a cause of numerous phototoxic and photoallergic reactions. The harmful effect of UVB radiation can also be intensified by UVA radiation.

Skin cells in particular receive a high proportion of DNA damage as a result of frequent UV exposure and therefore require particularly efficient and well-functioning repair systems. Only efficient DNA repair systems intrinsic to the skin are able to correct damage rapidly, as a result of which changes in the skin and a premature appearance of skin-aging phenomena can be prevented. Efficient DNA repair thus makes a decisive contribution to the maintenance of a healthy vital skin. Stimulation of and support to repair systems intrinsic to the skin by cosmetic-dermatological ingredients are therefore very important.

The high reliability during DNA replication, which is necessary in view of the large number of nucleotides which have to be copied per cell division, is on the one hand guaranteed by the specificity of the DNA polymerases which are involved in the replication (replicases), and on the other hand by the exonuclease activity thereof which is additionally present: they catalyze the cutting out of unsuitable nucleotides and their replacement by the correct ones immediately after de novo synthesis. The damage which arises by chemical action (by mutagens, some of which can squeeze between two superimposed base pairs of the double helix) and the changes caused by the action of radiation (where e.g. two superimposed thymine radicals can dimerize) is corrected by repair enzymes (including: endonucleases, polymerases, ligases), which cut the unsuitable nucleotides out of the DNA again and replace them. Some of these repair systems can be induced, i.e. they are only synthesized as required. The question as to which strand contains the original information and which has been synthesized incorrectly is apparently decided by the repair enzyme by reference to the methylation state.

Exposure to environmental influences (e.g. UV radiation, chemical and physical factors) causes damage to the DNA molecules of the cells of the body, but this can be corrected by DNA repair mechanisms intrinsic to the cell. The DNA damage is firstly identified, and then the damage present is repaired.

If the damage is not completely corrected shortly after it has arisen by repair processes, it accumulates in the form of permanent DNA damage in the cells and is subsequently passed on to the daughter cells. This permanent damage has, particularly as a result of possible long-term effects (stepwise losses of function), a high damage potential and plays an ever greater role in aging processes and in the triggering of damage even in childhood.

It was therefore an object of the present invention to remedy the shortcomings of the prior art. In particular, active ingredients and preparations are to be made available which ensure certain prophylaxis of damage to DNA intrinsic to the skin and/or, in a particular embodiment, serve to repair existing damage to DNA intrinsic to the skin.

Surprisingly, these objects are achieved by the use of folic acid for the preparation of cosmetic or dermatological preparations for the prophylaxis of damage to DNA intrinsic to the skin and/or for the repair of existing damage to DNA intrinsic to the skin.

According to the invention, folic acid and/or derivatives thereof have a high potential for the repair of damaged DNA. In addition, folic acid and/or derivatives thereof offer effective prophylaxis against DNA damage, in particular damage caused by UV radiation.

Folic acid has the following structure:

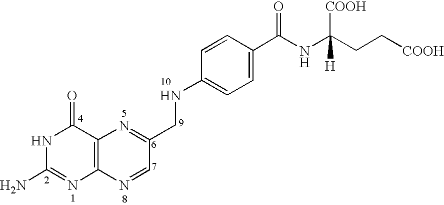

Folic acid occurs in liver, kidney, yeast, fungi, cereals and green leaves, primarily as a conjugate with poly-γ-L-glutamic acid (pteroylpolyglutamic acids). It was discovered as a growth substance for various microorganisms and has vitamin character for the human organisms. The daily requirement for adults is approximately 200 μg of bioavailable folate.

In the organism, folic acid is in equilibrium with 7,8-dihydrofolic acid (H₂folate; old abbreviation: FH₂) with participation by nicotinamide adenine dinucleotide phosphate and of the enzyme dihydrofolate reductase. H₂folate in turn arises in plants and a few microorganisms via a number of intermediate stages from guanosine-5′-triphosphate (guanosine phosphates) and converts, with the help of the dihydrofolate reductase, to (6S)-5,6,7,8-tetrahydrofolic acid, the actual physiologically effective form of folic acid.

7,8-Dihydrofolic acid has the following chemical structure:

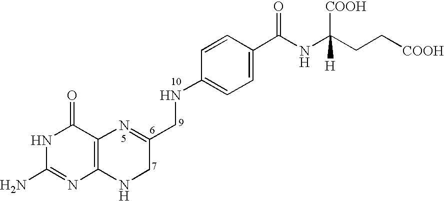

(6S)-5,6,7,8-Tetrahydrofolic acid has the following chemical structure:

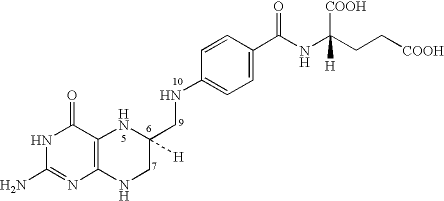

The term “derivatives of folic acid” is to be understood as meaning, in particular, the abovementioned dihydrofolic acid and tetrahydrofolic acid.

According to the invention, the cosmetic or dermatological preparations can have the customary composition and be used for the treatment, care and cleansing of the skin and/or the hair and as make-up product in decorative cosmetics. They preferably comprise 0.001% by weight to 10% by weight, preferably 0.05% by weight to 5% by weight, in particular 0.1-2.0% by weight, based on the total weight of the preparations, of folic acid and/or derivatives thereof used according to the invention.

According to the invention, it may be advantageous for the use according to the invention to add other substances which have the property to prevent damage to DNA intrinsic to the skin and/or to repair existing damage to DNA intrinsic to the skin.

However, according to the invention, it may also be advantageous, particularly if the use according to the invention relates to folic acid itself, and not to its derivatives, to manage without other such substances, namely flavonoids.

According to the invention, it is advantageous to add complexing agents to the folic acid and/or derivatives thereof used according to the invention, or to cosmetic or dermatological preparations comprising folic acid and/or derivatives thereof.

Complexing agents are auxiliaries used in cosmetics or medicinal pharmaceutical technology which are known per se. By complexing undesired metals such as Mn, Fe, Cu and others, it is possible, for example, to prevent undesired chemical reactions in cosmetic or dermatological preparations.

Complexing agents, in particular chelating agents, form complexes with metal atoms. In the presence of one or more polybasic complexing agents, i.e. chelating agents, these complexes are metallacycles. Chelates are compounds in which a single ligand occupies more than one co-ordination site on a central atom. In this case, normally extended compounds are thus closed as a result of complex formation via a metal atom or a metal ion to form rings. The number of bonded ligands depends on the co-ordination number of the central metal. A prerequisite for formation of the chelate is that the compound reacting with the metal contains two or more atomic groupings which act as electron donors.

The complexing agent(s) can advantageously be chosen from the group of customary compounds, preferably at least one substance from the group consisting of tartaric acid and anions thereof, citric acid and anions thereof, aminopolycarboxylic acids and anions thereof (such as, for example, ethylenediaminetetraacetic acid (EDTA) and anions thereof, nitrilotriacetic acid (NTA) and anions thereof, hydroxyethylenediaminotriacetic acid (HOEDTA) and anions thereof, diethyleneaminopentaacetic acid (DPTA) and anions thereof, trans-1,2-diaminocyclohexanetetraacetic acid (CDTA) and anions thereof).

According to the invention, the further complexing agent(s) is/are advantageously present in cosmetic or dermatological preparations preferably in amounts of from 0.01% by weight to 10% by weight, preferably from 0.05% by weight to 5% by weight, particularly preferably 0.1-2.0% by weight, based on the total weight of the preparations.

For use, according to the invention, the cosmetic and dermatological preparations are applied to the skin and/or the hair in an adequate amount in the customary manner for cosmetics.

Cosmetic and dermatological preparations according to the invention can be in various forms. Thus, they can, for example, be a solution, an anhydrous preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or else an aerosol. It is also advantageous to administer folic acid and/or derivatives thereof in encapsulated form, for example in collagen matrices and other customary encapsulation materials, for example as cellulose capsules, in gelatin, wax matrices or liposomally encapsulated. In particular, wax matrices, as are described in DE-A 43 08 282, have proven to be favorable.

For the purposes of the present invention, it is also possible and advantageous to incorporate folic acid and/or derivatives thereof into aqueous systems or surfactant preparations for cleansing the skin and the hair.

The cosmetic and dermatological preparations according to the invention, can comprise cosmetic auxiliaries such as are usually used in such preparations, for example preservatives, bactericides, perfumes, antifoams, dyes, pigments which have a coloring action, thickeners, surfactants, emulsifiers, softeners, moisturizers and/or humectants, fats, oils, waxes or other customary constituents of a cosmetic or dermatological preparation, such as alcohols, polyols, polymers, foam stabilizers, electrolytes, organic solvents or silicone derivatives.

In particular, folic acid and/or derivatives thereof can also be combined, according to the invention, with other antioxidants and/or free-radical scavengers.

It is also advantageous to add antioxidants to the preparations according to the invention. The antioxidants are advantageously chosen from the group consisting of amino acids (for example glycine, histidine, tyrosine, tryptophan) and derivatives thereof, imidazoles (for example urocanic acid) and derivatives thereof, peptides such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and furthermore (metal) chelating agents (for example α-hydroxy-fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic resin acid, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide) and the derivatives of these active ingredients which are suitable according to the invention (salts, esters, ethers, sugars, nucleotides, nucleosides, peptides and lipids).

The amount of the abovementioned antioxidants (one or more compounds) in the preparations is preferably from 0.001 to 30% by weight, particularly preferably 0.05-20% by weight, in particular 1-10% by weight, based on the total weight of the preparation.

If vitamin E and/or derivatives thereof is or are the additional antioxidant or antioxidants, it is advantageous to choose the respective concentrations thereof from the range 0.001-10% by weight, based on the total weight of the formulation.

If vitamin A or vitamin A derivatives or carotenes or derivatives thereof is or are the additional antioxidant or antioxidants, it is advantageous to choose the respective concentrations thereof from the range 0.001-10% by weight, based on the total weight of the preparation.

Emulsions according to the invention are advantageous and comprise, for example, said fats, oils, waxes and other fatty substances, and also water and an emulsifier, as is customarily used for this type of formulation.

The lipid phase can advantageously be chosen from the following group of substances:

mineral oils, mineral waxes;

oils, such as triglycerides of capric or of caprylic acid, also natural oils such as, for example, castor oil;

fats, waxes and other natural and synthetic fatty substances, preferably esters of fatty acids with alcohols of low C number, for example with isopropanol, propylene glycol or glycerol, or esters of fatty alcohols with alkanoic acids of low C number or with fatty acids;

alkyl benzoates;

silicone oils, such as dimethylpolysiloxanes, diethylpolysiloxanes, diphenylpolysiloxanes and mixed forms thereof.

For the purposes of the present invention, the oil phase of the emulsions, oleogels and hydrodispersions or lipodispersions is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then be advantageously chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, e.g. jojoba oil.

The oil phase can also advantageously be chosen from the group of branched and unbranched hydrocarbons and hydrocarbon waxes, silicone oils, dialkyl ethers, from the group of saturated or unsaturated, branched or unbranched alcohols, and also fatty acid triglycerides, namely the triglycerol esters of saturated and/or unsaturated, branched and/or unbranched alkanecarboxylic acids having a chain length of from 8 to 24, in particular 12-18, carbon atoms. The fatty acid triglycerides can advantageously be chosen, for example, from the group of synthetic, semi-synthetic and natural oils, e.g. olive oil, sunflower oil, soybean oil, groundnut oil, rapeseed oil, almond oil, palm oil, coconut oil, palm kernel oil and the like.

For the purposes of the present invention, any mixtures of such oil and wax components can also advantageously be used. When required, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂-C₁₅-alkyl benzoate, caprylic/capric triglyceride and dicaprylyl ether.

Mixtures of C₁₂-C₁₅-alkyl benzoate and 2-ethylhexyl isostearate, mixtures of C₁₂-C₁₅-alkyl benzoate and isotridecyl isononanoate and mixtures of C₁₂-C₁₅-alkyl benzoate, 2-ethylhexyl isostearate and isotridecyl isononanoate are particularly advantageous.

For the purposes of the present invention, of the hydrocarbons, paraffin oil, squalane and squalene can advantageously be used.

The oil phase can advantageously also contain cyclic or linear silicone oils or can consist entirely of such oils, although it is preferable to use an additional content of other oil phase components in addition to the silicone oil or silicone oils.

Cyclomethicone (octamethylcyclotetrasiloxane) is advantageously used as the silicone oil to be used according to the invention. However, other silicone oils can also be advantageously used for the purposes of the present invention, for example hexamethylcyclotrisiloxane, polydimethylsiloxane, poly(methylphenylsiloxane).

Mixtures of cyclomethicone and isotridecyl isononanoate and mixtures of cyclomethicone and 2-ethylhexyl isostearate are also particularly advantageous.

If appropriate, the aqueous phase of the preparations according to the invention advantageously comprises alcohols, diols or polyols of low C number and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, also alcohols of low C number, for example ethanol, isopropanol, 1,2-propanediol and glycerol, and, in particular, one or more thickeners, which can advantageously be chosen from the group consisting of silicon dioxide, aluminum silicates, polysaccharides and derivatives thereof, for example hyaluronic acid, xanthan gum and hydroxypropylmethylcellulose, particularly advantageously from the group of polyacrylates, preferably a polyacrylate from the group of Carbopols, for example Carbopols of types 980, 981, 1382, 2984 and 5984, in each case individually or in combination.

In particular, mixtures of the abovementioned solvents are used. In the case of alcoholic solvents, water may be a further constituent.

Emulsions according to the invention are advantageous and comprise, for example, said fats, oils, waxes and other fatty substances, and also water and an emulsifier, as is customarily used for this type of formulation.

Gels according to the invention customarily comprise alcohols of low C number, for example ethanol, isopropanol, 1,2-propanediol, glycerol, and water and/or an abovementioned oil in the presence of a thickener which, in the case of oily-alcoholic gels, is preferably silicon dioxide or an aluminum silicate, and in the case of aqueous-alcoholic or alcoholic gels, is preferably a polyacrylate.

Suitable propellants for preparations according to the invention which can be sprayed from aerosol containers are the customary known, readily volatile, liquefied propellants, for example hydrocarbons (propane, butane, isobutane), which may be used alone or in mixtures with one another. Compressed air can also be used advantageously.

Preparations according to the invention can advantageously also comprise substances which absorb UV radiation in the UVB region, the total amount of filter substances being, for example, from 0.1% by weight to 30% by weight, preferably from 0.5 to 10% by weight, in particular from 1.0 to 6.0% by weight, based on the total weight of the preparations, in order to provide cosmetic formulations which protect the skin or hair from the entire range of ultraviolet radiation. They can also be used as sunscreen compositions for hair or skin.

If the preparations according to the invention comprise UVB filter substances, these may be oil-soluble or water-soluble. Advantageous oil-soluble UVB filter substances are, for example:

3-benzylidenecamphor derivatives, preferably 3-(4-methylbenzylidene)camphor and 3-benzylidenecamphor;

4-aminobenzoic acid derivatives, preferably 2-ethylhexyl 4-(dimethylamino)benzoate and amyl 4-(dimethylamino)benzoate;

esters of cinnamic acid, preferably 2-ethylhexyl 4-methoxycinnamate and isopentyl 4-methoxycinnamate;

esters of salicylic acid, preferably 2-ethylhexyl salicylate, 4-isopropylbenzyl salicylate and homomenthyl salicylate,

derivatives of benzophenone, preferably 2-hydroxy-4-methoxybenzophenone, 2-hydroxy-4-methoxy-4′-methylbenzophenone and 2,2′-dihydroxy-4-methoxy-benzophenone;

esters of benzalmalonic acid, preferably di(2-ethylhexyl) 4-methoxybenzalmalonate and

2,4,6-tris(p-2-ethylhexoxy carbonylanilino)-1,3,5-triazine.

Advantageous water-soluble UVB filters are, for example:

salts of 2-phenylbenzimidazole-5-sulfonic acid, such as its sodium, potassium or its triethanolammonium salt, and the sulfonic acid itself;

sulfonic acid derivatives of benzophenones, preferably 2-hydroxy-4-methoxybenzophenone-5-sulfonic acid and its salts;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and their salts, and also 1,4-di(2-oxo-10-sulfo-3-bornylidenemethyl)benzene and its salts (the corresponding 10-sulfato compounds, for example the corresponding sodium, potassium or triethanolammonium salt) also referred to as benzene-1,4-di(2-oxo-3-bornylidenemethyl)-10-sulfonic acid.

The list of the UVB filters mentioned which can be used in combination with folic acid and/or derivatives thereof, is not of course intended to be limiting.

The invention also provides for the use of a combination of folic acid and/or derivatives thereof with at least one UVB filter and for the use of a combination of folic acid and/or derivatives thereof with at least one UVB filter in a cosmetic or dermatological preparation.

It can also be advantageous to combine folic acid and/or derivatives thereof used according to the invention with UVA filters which have to date customarily been present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. These combinations and preparations comprising these combinations are also provided by the invention. The amounts used are as for the UVB combination.

The invention also provides for the use of a combination of folic acid and/or derivatives thereof with at least one UVA filter as an antioxidant and for the use of a combination of folic acid and/or derivatives thereof with at least one UVA filter as an antioxidant in a cosmetic or dermatological preparation.

The invention also provides for the use of a combination of folic acid and/or derivatives thereof with at least one UVA filter and at least one UVB filter as an antioxidant and for the use of a combination of folic acid and/or derivatives thereof with at least one UVA filter and at least one UVB filter as an antioxidant in a cosmetic or dermatological preparation.

Cosmetic and dermatological preparations with an effective content of folic acid and/or derivatives thereof can also contain inorganic pigments which are normally used in cosmetics for protecting the skin against UV rays. These are oxides of titanium, zinc, zirconium, silicon, manganese, cerium and mixtures thereof, and modifications in which the oxides are the active agents. Particular preference is given to pigments based on titanium dioxide.

These combinations of UVA filter and pigment and preparations which comprise this combination are also provided by the invention. The quantities used may be as stated for the aforementioned combinations.

Cosmetic and dermatological preparations for protecting the hair against UV rays according to the invention are, for example, shampoos, preparations which are applied to the hair when rinsing the hair before or after shampooing, before or after permanent waving, before or after coloring or bleaching, preparations for blow-drying or setting the hair, preparations for coloring or bleaching, a styling and treatment lotion, a hairspray or a permanent wave solution.

The cosmetic and dermatological preparations comprise active ingredients and auxiliaries as are usually used for this type of preparation for hair care and hair treatment. Auxiliaries include preservatives, surface-active substances, antifoams, thickeners, emulsifiers, fats, oils, waxes, organic solvents, bactericides, perfumes, dyes or pigments whose task is to color the hair or the cosmetic or dermatological preparation itself, electrolytes and anti-grease substances.

For the purposes of the present invention, electrolytes are understood as meaning water-soluble alkali metal, ammonium, alkaline earth metal (including magnesium) and zinc salts of inorganic anions and any mixtures of such salts, it being necessary to ensure that these salts are pharmaceutically or cosmetically safe.

The anions according to the invention are preferably chosen from the group consisting of chlorides, sulfates and hydrogensulfates, phosphates, hydrogenphosphates and linear and cyclic oligophosphates and carbonates and hydrogencarbonates.

Cosmetic preparations in the form of a skin cleanser or shampoo preferably comprise at least one anionic, nonionic or amphoteric surface-active substance, or else mixtures of such substances, the folic acid and/or derivatives thereof in aqueous medium, and auxiliaries usually used for this purpose. The surface-active substance and the mixtures of these substances can be present in the shampoo in a concentration of between 1% by weight and 50% by weight.

If the cosmetic or dermatological preparations are in the form of a lotion which is rinsed out and applied, for example, before or after bleaching, before or after shampooing, between two shampooing steps, before or after permanent waving, they are for example, aqueous or aqueous-alcoholic solutions optionally comprising surface-active substances in a concentration of between 0.1 and 10% by weight, preferably between 0.2 and 5% by weight.

These cosmetic or dermatological preparations can also be in the form of aerosols with the auxiliaries usually used for this purpose.

A cosmetic preparation in the form of a lotion which is not rinsed out, in particular a lotion for setting the hair, a lotion which is used for blow drying the hair, a styling and treatment lotion, is generally in the form of an aqueous, alcoholic or aqueous-alcoholic solution, and contains at least one cationic, anionic, nonionic or amphoteric polymer or also mixtures thereof, and also folic acid and/or derivatives thereof in an effective concentration. The amount of polymers used is, for example, between 0.1 and 10% by weight, preferably between 0.1 and 3% by weight.

Cosmetic preparations for treating and caring for the hair which comprise folic acid and/or derivatives thereof can be in the form of emulsions which are of the nonionic or anionic type. Nonionic emulsions comprise, in addition to water, oils or fatty alcohols which may, for example, also be polyethoxylated or polypropoxylated, or also mixtures of the two organic components. These emulsions optionally contain cationic surface-active substances.

According to the invention, cosmetic preparations for treating and caring for the hair can be in the form of gels which, in addition to an effective content of active ingredients according to the invention and solvents usually used therefor, preferably water, also contain organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, for example aluminum silicates such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The amount of folic acid and/or derivatives thereof in a product intended for hair is preferably from 0.05% by weight to 10% by weight, in particular from 0.5% by weight to 5% by weight, based on the total weight of the product.

Aqueous cosmetic cleansers according to the invention or low-water or water-free cleanser concentrates intended for aqueous cleansing may comprise anionic, nonionic and/or amphoteric surfactants, for example

conventional soaps, e.g. fatty acid salts of sodium

alkyl sulfates, alkyl ether sulfates, alkanesulfonates and alkylbenzenesulfonates

sulfoacetates

sulfobetaines

sarcosinates

amidosulfobetaines

sulfosuccinates

sulfosuccinic acid monoesters

alkyl ether carboxylates

protein-fatty acid condensates

alkylbetaines and amidobetaines

fatty acid alkanolamides

polyglycol ether derivatives

Cosmetic preparations which are cosmetic skin cleansing preparations can be in liquid or solid form. In addition to folic acid and/or derivatives thereof, they preferably comprise at least one anionic, nonionic or amphoteric surface-active substance or mixtures thereof, if desired one or more electrolytes and auxiliaries usually used for this purpose. The surface-active substance can be present in the cleansing preparations in a concentration of between 1 and 94% by weight, based on the total weight of the preparations.

Cosmetic preparations in the form of a shampoo preferably comprise, in addition to an effective amount of folic acid and/or derivatives thereof, at least one anionic, nonionic or amphoteric surface-active substance or mixtures thereof, if desired one electrolyte according to the invention and auxiliaries which are usually used for this purpose. The surface-active substance can be present in the shampoo in a concentration of between 1% by weight and 94% by weight.

The compositions according to the invention comprise, apart from the aforementioned surfactants, water and, when required, the additives customary in cosmetics, for example perfume, thickeners, dyes, deodorants, antimicrobial substances, refatting agents, complexing agents and sequestering agents, pearlizing agents, plant extracts, vitamins, active ingredients and the like.

The present invention also covers a cosmetic method of protecting the skin and hair against oxidative or photooxidative processes which comprises applying a cosmetic composition which comprises an effective concentration of folic acid and/or derivatives thereof in a sufficient quantity to the skin or hair.

The present invention likewise also covers a method of protecting cosmetic or dermatological preparations against oxidation or photooxidation, these preparations being, for example, preparations for the treatment and care of hair, in particular hair colorants, hairsprays, shampoos, color shampoos, and also make-up products such as, for example, nail varnishes, lipsticks, foundations, washing and shower preparations, creams for the treatment or care of the skin or all other cosmetic preparations whose constituents may be associated with stability problems because of oxidation or photooxidation during storage, wherein the cosmetic preparations have an effective content of folic acid and/or derivatives thereof.

The amount of folic acid and/or derivatives thereof in these preparations is preferably 0.01-10% by weight, preferably 0.05-5% by weight, in particular 0.1-2.0% by weight, based on the total weight of the preparations.

The invention also provides the process for the preparation of the cosmetic compositions according to the invention, which comprises incorporating folic acid and/or derivatives thereof into cosmetic or dermatological formulations in a manner known per se.

The examples below serve to illustrate the present invention without limiting it. Unless stated otherwise, all quantities, proportions and percentages are by weight and based on the total amount or on the total weight of the preparations.

### EXAMPLE 1

### O/W Cream

| | % by wt. |
|---|---|
| Glyceryl stearate citrate | 2.00 |
| Stearyl alcohol | 5.00 |
| Caprylic/capric triglycerides | 4.00 |
| Octyldodecanol | 4.00 |
| Glycerol | 3.00 |
| Carbomer | 0.10 |
| Folic acid | 0.30 |
| EDTA | 0.10 |
| Sodium hydroxide | q.s. |
| Preservative | q.s. |
| Perfume | q.s. |
| Water, demineralized | ad 100.00 |
| pH adjusted to 6.00 | |

### EXAMPLE 2

### O/W Cream

| | % by wt. |
|---|---|
| Glyceryl stearate SE | 4.00 |
| PEG-40 stearates | 1.00 |
| Cetyl alcohol | 3.00 |
| Caprylic/capric triglycerides | 5.00 |
| Paraffin oil | 5.00 |
| Glycerol | 3.00 |
| Carbomer | 0.10 |
| Folic acid | 0.10 |
| EDTA | 0.10 |
| Sodium hydroxide | q.s. |
| Preservative | q.s. |
| Perfume | q.s. |
| Water, demineralized | ad 100.00 |
| pH adjusted to 7.0 | |

### EXAMPLE 3

### O/W Cream

| | % by wt. |
|---|---|
| Glyceryl stearate SE | 3.00 |
| Stearic acid | 1.00 |
| Cetyl alcohol | 2.00 |
| Dicaprylyl ether | 4.00 |
| Caprylic/capric triglycerides | 3.00 |
| Paraffin oil | 2.00 |
| Glycerol | 3.00 |
| Butylene glycol | 3.00 |
| Carbomer | 0.10 |
| Folic acid | 1.00 |
| Sodium hydroxide | q.s. |
| Preservative | q.s. |
| Perfume | q.s. |
| Water, demineralized | ad 100.00 |
| pH adjusted to 7.5 | |

### EXAMPLE 4

### O/W Lotion

| | % by wt. |
|---|---|
| Glyceryl stearate, Ceteth-20 | 1.00 |
| Sorbitan stearate | 1.00 |
| Stearyl alcohol | 1.00 |
| Caprylic/capric triglycerides | 2.00 |
| Paraffin oil | 4.00 |
| Glycerol | 3.00 |
| Carbomer | 0.10 |
| Folic acid | 0.50 |
| Tocopherol | 0.05 |
| Sodium hydroxide | q.s. |
| Preservative | q.s. |
| Perfume | q.s. |
| Water, demineralized | ad 100.00 |
| pH adjusted to 5.5 | |

### EXAMPLE 5

### W/O Cream

| | % by wt. |
|---|---|
| Triglycerol diisostearate | 3.50 |
| Glycerol | 3.00 |
| Polyglyceryl-2 polyhydroxystearate | 3.50 |
| Folic acid | 0.10 |
| Magnesium sulfate | 0.60 |
| Isopropyl stearate | 2.00 |
| Dicaprylyl ether | 8.00 |
| Cetearyl isononanoate | 6.00 |
| Preservative | q.s. |
| Perfume | q.s. |
| Water, demin. | ad 100.00 |

### EXAMPLE 6

### Emulsion Make-up

| | % by wt. |
|---|---|
| Glyceryl stearate SE | 5.00 |
| Stearyl alcohol | 2.00 |
| Dimethicone | 2.00 |
| Glycerol | 3.00 |
| Carbomer | 0.15 |
| Mica | 1.00 |
| Magnesium silicate | 1.00 |
| Iron oxide | 1.00 |
| Titanium dioxide | 2.50 |
| Talc | 5.00 |
| Folic acid | 1.00 |
| Sodium hydroxide | q.s. |
| Preservative | q.s. |
| Perfume | q.s. |
| Water, demineralized | ad 100.00 |
| pH adjusted to 6.0 | |

### EXAMPLE 7

### W/O/W Cream

| | % by wt. |
|---|---|
| Glyceryl stearate | 3.00 |
| PEG-100 stearate | 0.75 |
| Behenyl alcohol | 2.00 |
| Caprylic/capric triglycerides | 8.00 |
| Octyldodecanol | 5.00 |
| C₁₂₋₁₅-alkyl benzoate | 3.00 |
| Panthenol | 3.00 |
| BHT | 0.05 |
| Magnesium sulfate (MgSO₄) | 0.80 |
| EDTA | 0.10 |
| Folic acid | 0.10 |
| Preservative | q.s. |
| Perfume | q.s. |
| Water, demineralized | ad 100.00 |
| pH adjusted to 6.0 | |

### EXAMPLE 8

### Hydrodispersion Gel

| | % by wt. |
|---|---|
| Carbomer | 0.40 |
| Xanthan gum | 0.20 |
| Cetylstearyl alcohol | 2.00 |
| C₁₂₋₁₅-alkyl benzoates | 5.00 |
| Caprylic/capric triglycerides | 3.00 |
| Glycerol | 3.00 |
| Folic acid | 0.30 |
| Sodium hydroxide | q.s. |
| Preservative | q.s. |
| Perfume | q.s. |
| Water, demineralized | ad 100.00 |
| pH adjusted to 6.5 | |

1. A method of preventing damage to DNA intrinsic to skin and/or for repairing existing damage to DNA intrinsic to skin, said method comprising topically applying to skin an effective amount therefor of at least one of folic acid or a derivative thereof.

2. The method according to claim 1, which comprises topically applying to skin a cosmetic or topical dermatological preparation comprising folic acid and/or a derivative thereof in a concentration of 0.01-10% by weight based on the total weight of the preparation.

3. The method according to claim 2, which comprises topically applying to skin a cosmetic or topical dermatological preparation comprising folic acid and/or a derivative thereof in a concentration of 0.05-5% by weight based on the total weight of the preparation.

4. The method according to claim 3, which comprises topically applying to skin a cosmetic or topical dermatological preparation comprising folic acid and/or a derivative thereof in a concentration of 0.1-2% by weight based on the total weight of the preparation.

5. A cosmetic preparation comprising dihydrofolic acid and/or tetrahydrofolic acid.

6. The preparation according to claim 5, which comprises dihydrofolic acid and/or tetrahydrofolic acid in a concentration of 0.01-10% by weight based on the total weight of the preparation.

7. The preparation according to claim 6, which comprises dihydrofolic acid and/or tetrahydrofolic acid in a concentration of 0.05-5% by weight based on the total weight of the preparation.

8. The preparation according to claim 7, which comprises dihydrofolic acid and/or tetrahydrofolic acid in a concentration of 0.1-2% by weight based on the total weight of the preparation.
